(19) United States
(12) Patent Application Publication
(10) Pub. No.: US 2013/0096410 A1
(43) Pub. Date: Apr. 18, 2013

(54) BIOSIGNAL PROCESSING APPARATUS, ELECTROENCEPHALOGRAPH, AND BIOSIGNAL PROCESSING METHOD

(71) Applicant: Yusaku Nakashima, Tokyo (JP)

(72) Inventor: Yusaku Nakashima, Tokyo (JP)

(73) Assignee: Sony Corporation, Tokyo (JP)

(21) Appl. No.: 13/613,819

(22) Filed: Sep. 13, 2012

(30) Foreign Application Priority Data
Oct. 12, 2011 (JP) 2011-224605

(51) Int. Cl. A61B 5/0478; A61B 5/0476

(52) U.S. Cl. 600/383; 600/544

(57) ABSTRACT

A biosignal processing apparatus includes a signal acquisition unit and a determination unit. The signal acquisition unit is configured to acquire an output signal of an electrode that is placed on a biological surface. The determination unit is connected to the signal acquisition unit to determine, based on first frequency characteristics being frequency characteristics of the output signal, whether the electrode is in a first state in which the electrode is electrically connected to a measurement site of the biological surface or in a second state in which the electrode is electrically disconnected to the measurement site.

### BACKGROUND

The present disclosure relates to a biosignal processing apparatus, an electroencephalograph, and a biosignal processing method that determine an electrical connection state between a biological surface and an electrode.

From the past, acquiring biosignals such as brain waves and myogenic potentials by electrodes placed on measurement sites of a biological surface has been widely performed. Here, the electrodes and the biological surface need to be electrically connected to each other. However, there has been a problem that it is difficult to keep the electrodes and the biological surface in an appropriate connection state, for example, due to the presence of the hair and the like and non-uniformity of the shape of the biological surface. In addition, regarding a determination of the electrical connection state, there are not only cases where it is easy to make a determination, for example, a detachment of an electrode that occurs due to a motion of a living body or the like, but also cases where it is difficult to make a determination, for example, an intervention of the hair.

In view of this, for example, an apparatus that detects an electrical connection state between an electrode that detects a brain wave signal and a scalp has been considered. Japanese Patent Application Laid-open No. 2006-212348 (hereinafter, referred to as Patent Document 1) describes an apparatus for detecting a contact of a brain wave electrode. This apparatus includes coils provided near brain wave electrodes held in contact with a scalp. This apparatus detects an electrical connection state between the scalp and each of the brain wave electrodes based on whether or not an induced current that generates upon applying a current to each coil flows to the scalp through each brain wave electrode.

### SUMMARY

However, with the apparatus for detecting a contact of a brain wave electrode described in Patent Document 1, it is necessary to provide each electrode with an electric circuit that applies a current to each coil in addition to an electric circuit for brain wave measurement, which makes the apparatus configuration complex. Further, while the connection state is being detected, it is necessary to stop the brain wave measurement. Therefore, in the case where a continuous brain wave measurement for a long period of time, for example, sleep hours, is necessary, it is difficult to detect the connection state for that period.

In view of the above-mentioned circumstances, there is a need for providing a biosignal processing apparatus and an electroencephalograph that have a simple configuration and are capable of detecting an electrical connection state between a measurement site of a biological surface and an electrode even while acquiring an output signal, and a biosignal processing method.

According to an embodiment of the present disclosure, there is provided a biosignal processing apparatus including a signal acquisition unit and a determination unit.

The signal acquisition unit is configured to acquire an output signal of an electrode that is placed on a biological surface.

The determination unit is connected to the signal acquisition unit to determine, based on first frequency characteristics being frequency characteristics of the output signal, whether the electrode is in a first state in which the electrode is electrically connected to a measurement site of the biological surface or in a second state in which the electrode is electrically disconnected to the measurement site.

With this configuration, by using the fact that an output signal having frequency characteristics different from those of an original biosignal generates, for example, when the electrical connection between the biological surface and the electrode breaks due to a intervention of the hair, the air, or the like, it is possible to determine an electrical connection state between the biological surface and the electrode. Accordingly, without making the apparatus configuration complex, it is possible to determine the connection state while continuing acquiring the output signal.

The determination unit may be configured to compare the first frequency characteristics with second frequency characteristics being frequency characteristics of one of colored noise and white noise and to determine, when the first frequency characteristics are different from the second frequency characteristics, that the electrode is in the first state and determine, when the first frequency characteristics are similar to the second frequency characteristics, that the electrode is in the second state.

When the electrical connection between the biological surface and the electrode breaks, the colored noise such as pink noise or the white noise may generate as the output signal, for example. Therefore, with this configuration, based on the frequency characteristics of such noise, it is possible to easily determine the electrical connection state between the biological surface and the electrode.

The determination unit may be configured to detect intensity with respect to a specific frequency and to determine, when the intensity is smaller than a predetermined threshold, that the electrode is in the first state and determine, when the intensity is equal to or larger than the threshold, that the electrode is in the second state.

When the electrical connection between the biological surface and the electrode breaks, the output signal having larger intensity due to noise or the like than in the case where the electrical connection is established may generate. Therefore, with this configuration, by comparing the intensity at the specific frequency with the predetermined threshold, it is possible to easily determine the electrical connection state between the biological surface and the electrode.

The biosignal processing apparatus may further include an output unit that is connected to the determination unit to be capable of outputting a determination result of the determination unit.

The output unit makes it possible to transmit the determination result to an external apparatus or the like.

The biosignal processing apparatus may further include a warning unit that is connected to the determination unit to be activated when the determination result is that the electrode is in the second state.

The warning unit makes it possible to warn, when the electrode is in the second state in which the electrical connection between the electrode and the biological surface breaks, a measurer, a user, or the like of that.

The biosignal processing apparatus may further include a measurement unit configured to monitor, when the determination unit determines that the electrode is in the first state, the output signal as a biosignal over time.

With this configuration, when the determination unit determines that an appropriate biosignal is acquired, it is possible to continue monitoring by the measurement unit. Accordingly, it is possible to improve reliability of acquired data.

Specifically, the determination unit may be configured to perform Fourier transform on the output signal, to thereby acquire the first frequency characteristics.

Thus, the determination unit can easily acquire the first frequency characteristics from the output signal.

According to an embodiment of the present disclosure, there is provided a biosignal processing apparatus including an electrode, a signal acquisition unit, and a determination unit.

The electrode is placed on a biological surface.

The signal acquisition unit is configured to acquire an output signal of the electrode.

The determination unit is connected to the signal acquisition unit to determine, based on first frequency characteristics being frequency characteristics of the output signal, whether the electrode is in a first state in which the electrode is electrically connected to a measurement site of the biological surface or in a second state in which the electrode is electrically disconnected to the measurement site.

This configuration allows the biosignal processing apparatus to include the electrode for acquiring the biosignal without making the apparatus configuration complex.

According to an embodiment of the present disclosure, there is provided an electroencephalograph including a head accessory, a signal acquisition unit, and a determination unit.

The head accessory is configured to place an electrode on a head surface of a user.

The signal acquisition unit is configured to acquire an output signal of the electrode.

The determination unit is connected to the signal acquisition unit to determine, based on first frequency characteristics being frequency characteristics of the output signal, whether the electrode is in a first state in which the electrode is electrically connected to a measurement site of the biological surface or in a second state in which the electrode is electrically disconnected to the measurement site.

The head is a site especially difficult to establish an appropriate electrical connection due to the presence of the hair, a curved surface thereof, and the like. In addition, it is desirable to perform a measurement for a relatively long period of time, for example, sleep hours. Even under such circumstances, the electroencephalograph is able to easily grasp a point of time when the electrical connection breaks, which can improve reliability of measured data.

According to an embodiment of the present disclosure, there is provided a biosignal processing method including acquiring an output signal through an electrode for acquiring a biosignal of a biological surface.

Whether the electrode is in a first state in which the electrode is electrically connected to a measurement site of the biological surface or in a second state in which the electrode is electrically disconnected to the measurement site is determined based on first frequency characteristics being frequency characteristics of the output signal.

The determining may include determining whether the electrode is in the first state or in the second state while monitoring the acquired output signal.

With this configuration, a real-time determination of the connection state between the biological surface and the electrode is realized, and hence it is possible to take an appropriate measure, for example, reestablishment of the connection.

As described above, according to the embodiments of the present disclosure, it is possible to provide a biosignal processing apparatus and an electroencephalograph that have a simple configuration and are capable of detecting an electrical connection state between a measurement site of a biological surface and an electrode even while acquiring an output signal, and a biosignal processing method.

These and other objects, features and advantages of the present disclosure will become more apparent in light of the following detailed description of best mode embodiments thereof, as illustrated in the accompanying drawings.

### BRIEF DESCRIPTION OF DRAWINGS

FIG. 1 is a perspective view showing a state in which a biosignal processing apparatus (electroencephalograph) according to a first embodiment is attached to a user;

FIG. 2 is a schematic view showing a functional configuration of the biosignal processing apparatus (electroencephalograph) according to the first embodiment;

FIG. 3 is a flowchart showing an operation example of the biosignal processing apparatus (electroencephalograph) according to the first embodiment;

FIG. 4 is a graph showing an example of frequency characteristics of an output signal in a state (first state) in which electrodes and measurement sites of a head surface of the user are electrically connected to each other, in which the horizontal axis indicates a frequency and the vertical axis indicates intensity;

FIG. 5 is a graph showing an example of frequency characteristics of an output signal in a state (second state) in which the electrodes and the measurement sites of the head surface of the user are electrically disconnected to each other, in which the horizontal axis indicates a frequency and the vertical axis indicates intensity;

FIG. 6 is a schematic graph showing frequency characteristics of pink noise, in which the horizontal axis indicates a frequency and the vertical axis indicates intensity;

FIG. 7 is a schematic view showing a functional configuration of a biosignal processing apparatus (electroencephalograph) according to a second embodiment;

FIG. 8 is a flowchart showing an operation example of the biosignal processing apparatus (electroencephalograph) according to the second embodiment;

FIG. 9 is a schematic diagram showing a functional configuration of a biosignal processing apparatus (electroencephalograph) according to a third embodiment; and

FIG. 10 is a flowchart showing an operation example of the biosignal processing apparatus (electroencephalograph) according to the third embodiment.

### DETAILED DESCRIPTION OF EMBODIMENTS

Hereinafter, embodiments of the present disclosure will be described with reference to the drawings.

### First Embodiment

### [Configuration of Biosignal Processing Apparatus]

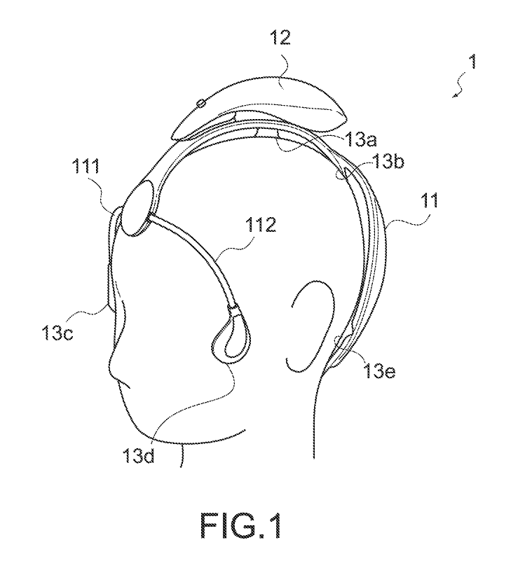

FIG. 1 is a perspective view showing a state in which a biosignal processing apparatus (electroencephalograph) according to this embodiment is attached to a user. The electroencephalograph 1 includes a headgear (head accessory) 11 and a casing 12. The headgear 11 is provided with electrodes 13a to 13e on an opposite side of the user. The casing 12 is detachably connected to the headgear 11 and houses electrical components, which will be described later.

The headgear 11 is configured so that the electrodes 13a to 13e can be placed on the head surface of the user. The headgear 11 is constituted of a member extending from the forehead of the user through the parietal region to the occipital region. Further, the headgear 11 can be formed corresponding to the head shape of the user and the arrangement of the electrodes. For example, the headgear 11 may include arms 111 and 112 or the like for arranging the electrodes 13c and 13d and the like in place, which will be described later. Further, the headgear 11 is formed of an elastic material, for example, a synthetic resin and supported on the head of the user by its elastic force.

The electrodes 13a to 13e are various electrodes to be provided to the headgear 11 and are arranged corresponding to predetermined measurement sites. For example, the parietal-region electrode 13a, the occipital-region electrode 13b, the right EOG (electrooculogram) electrode 13c, the left EOG electrode 13d, and the reference electrode 13e can be provided.

More specifically, the parietal-region electrode 13a and the occipital-region electrode 13b measure brain waves (EEG: electroencephalogram) of the user. Further, the right EOG electrode 13c is an electrode to be brought into contact with the right temple of the user and the left EOG electrode 13d is an electrode to be brought into contact with left temple of the user. These electrodes measure eye movements (EOG) of the user. The reference electrode 13e is an electrode for acquiring a potential (reference potential) to be a reference for measurement potentials from the electrodes 13a to 13d. For example, the reference electrode 13e is brought into contact with the back of the earlobe of the user. It should be noted that the names and arrangement of the electrodes and the like are merely examples and can be appropriately selected depending on needs.

Although the shape of the electrodes 13a to 13e is not particularly limited, brush-shaped electrodes made of a conductive material may be adopted, for example. Such a configuration enables the electrodes to push through the hair and be brought into contact with the head surface. Further, by constituting portions of the electrodes 13a to 13e, which are to abut against the biological surface, of a material having a liquid-holding capacity, such as felt, it is possible to provide an electrolytic solution or the like between the head surface and the electrodes 13a to 13e. With this, without fixing the head surface and the electrodes to each other with paste or the like, it is possible to ensure electrical connections between the measurement sites of the head surface and the electrodes 13a to 13e. In addition, it becomes easy to attach/detach the electrodes 13a to 13e thereto/therefrom.

The casing 12 is detachably connected to the headgear 11 as described above. The position in which the casing 12 is to be provided only needs to be a position not to interfere with the attachment of the headgear 11 and the motion of the user. For example, the position may be a position in the headgear 11 near the parietal region.

The casing 12 houses the electrical components such as a processor, a memory, and a communication interface that constitute a signal acquisition unit, a determination unit, and the like, which will be described later. The headgear 11 is provided with a wiring (not shown). The wiring connects the electrodes and the electrical components thereof to each other.

[Functional Configuration of Biosignal Processing Apparatus]

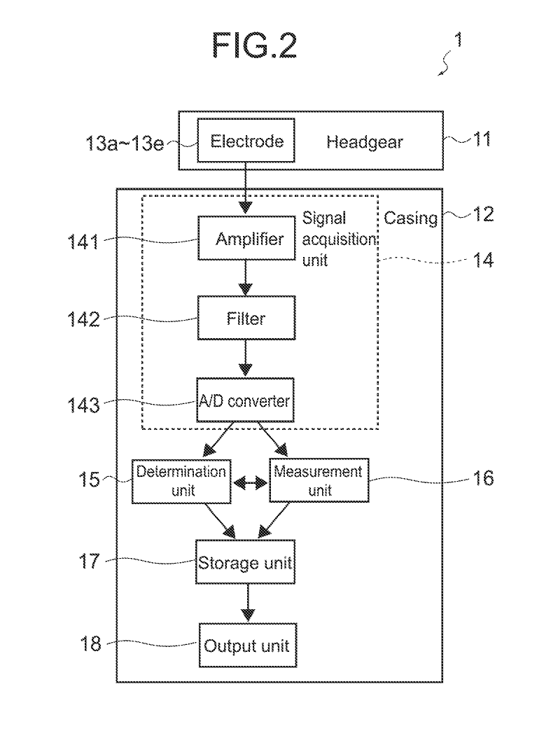

FIG. 2 is a schematic view showing a functional configuration of the electroencephalograph 1. As shown in the figure, the electroencephalograph 1 includes the headgear 11, the casing 12, the electrodes 13a to 13e, a signal acquisition unit 14, a determination unit 15, a measurement unit 16, a storage unit 17, and an output unit 18. Among them, the signal acquisition unit 14, the determination unit 15, the measurement unit 16, the storage unit 17, and the output unit 18 are all housed in the casing 12.

Output signals acquired by the electrodes 13a to 13e of the headgear 11 arrive at the signal acquisition unit 14 within the casing 12 through the wiring or the like. The signal acquisition unit 14 includes, for example, an amplifier 141, a filter 142, and an A/D (analog/digital) converter 143. The electrodes 13a to 13e are connected to the amplifier 141. The amplifier 141 is connected to the filter 142. The filter 142 is connected to the A/D converter 143. The A/D converter 143 is connected to each of the determination unit 15 and the measurement unit 16. Each of the determination unit 15 and the measurement unit 16 is connected to the storage unit 17. The storage unit 17 is connected to the output unit 18. In FIG. 2, for the sake of description, the electrodes 13a to 13e are shown as a single block. However, actually, each of the electrodes 13a to 13e is connected to the amplifier 141 through the wiring or the like.

The amplifier 141 amplifies the output signals. In the filter 142, a predetermined frequency bandwidth to be a measurement target is set and signal components other than this frequency bandwidth are removed. The A/D converter 143 converts the output signals into digital signals.

By performing Fourier transform on the output signals from the electrodes 13a to 13e, the determination unit 15 acquires the frequency characteristics of the output signals. In addition, based on the frequency characteristics (first frequency characteristics) of each of the output signals, electrical connection states between the electrodes 13a to 13e and the measurement sites of the head surface of the user are determined, respectively. As a determination method according to this embodiment, as will be described later, the connection states are determined by comparing frequency characteristics (second frequency characteristics) of pink noise with the first frequency characteristics.

The measurement unit 16 performs data processing such as montage processing (output of difference between measurement electrode and reference electrode) on the output signal. With this, for example, the output signals from the electrodes 13a to 13d are processed as time-series data on potential differences with the electrode 13e being the reference electrode. It should be noted that “monitoring” refers to acquiring the time-series data on potential differences between the output signal of the electrode 13e and the output signals from the electrodes 13a to 13d. The measurement unit 16 is connected to the determination unit 15 and is capable of acquiring a determination result in the determination unit 15.

The storage unit 17 is constituted of, for example, a flash memory. Data acquired by the determination unit 15 and the measurement unit 16 is temporarily stored on the storage unit 17.

The output unit 18 is constituted of, for example, a communication interface (communication IF). The data stored on the storage unit 17 is transmitted to an external apparatus or the like through the output unit 18 depending on needs. An output method is not particularly limited and a wireless or wired output method may be adopted.

As described above, the electroencephalograph 1 is configured to be capable of acquiring biosignals from the user and determining the connection states between the electrodes 13a to 13e and the measurement sites. It should be noted that such a functional configuration of the casing 12 is merely an example and a different configuration may be adopted.

[Operation of Electroencephalograph]

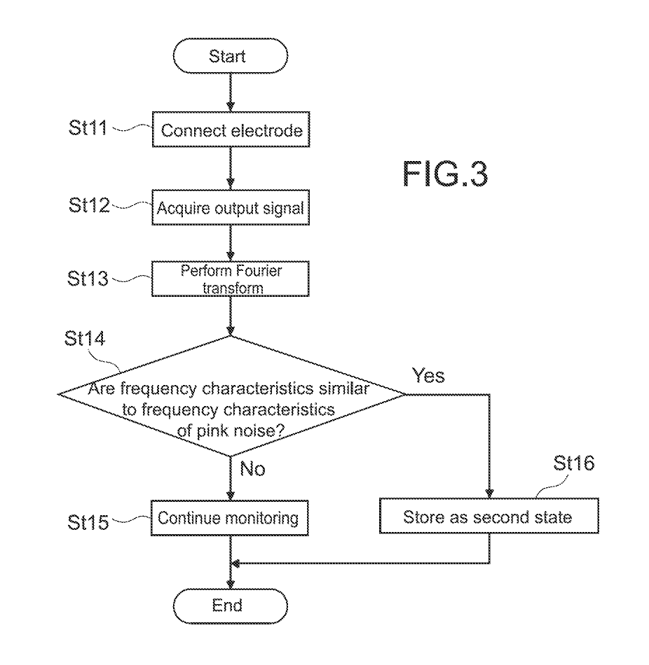

FIG. 3 is a flowchart showing an operation example of the electroencephalograph 1 according to this embodiment. Hereinafter, respective steps (St) shown in this flowchart will be described.

First, the electrodes 13a to 13e are placed on the head surface of the user (St11). In this state, the electroencephalograph 1 is activated.

Then, the output signals from the electrodes 13a to 13e placed on the head surface of the user are acquired by the signal acquisition unit 14 (St12). The output signals are first amplified by the amplifier 141. Then, the signal components of the output signals other than the predetermined frequency bandwidth are removed by the filter 142. In addition, the remaining signal components are converted by the A/D converter 143 into the digital signals. The thus processed output signals are supplied to the determination unit 15 and the measurement unit 16.

In the measurement unit 16, the acquired output signals are monitored over time (not shown). Further, a monitoring result can be stored on the storage unit 17 and then transmitted to the external apparatus or the like through the output unit 18.

The determination unit 15 performs Fourier transform on each of the output signals from the electrodes 13a to 13d (St13). As the Fourier transform, for example, high-speed Fourier transform may be performed. Accordingly, it is possible to easily acquire data on the spectral density (intensity) of each frequency component with respect to the output signal, that is, the first frequency characteristics. Further, the acquired data is smoothed depending on needs.

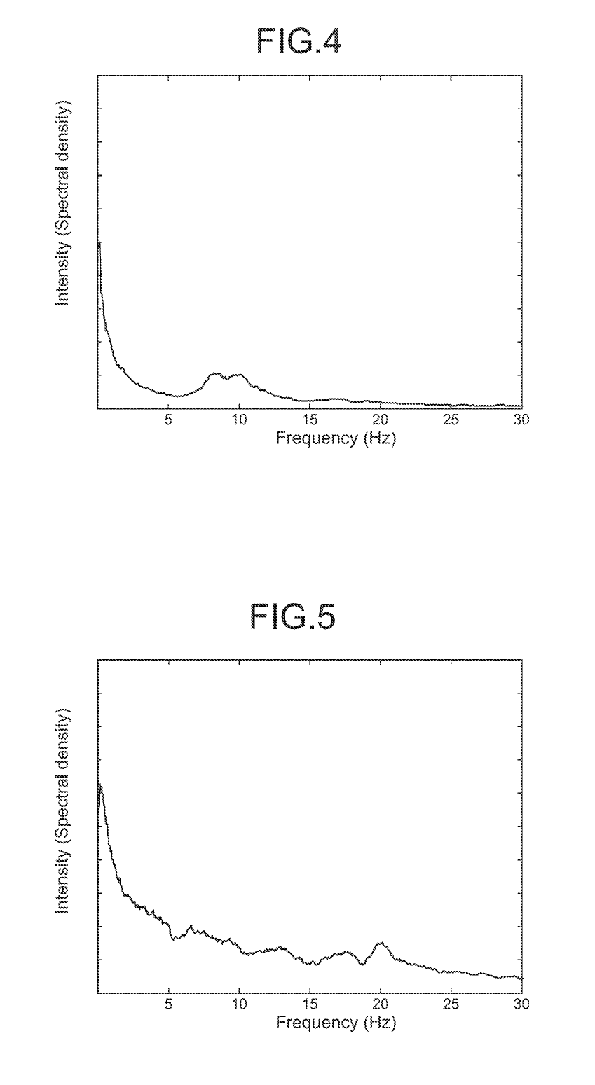

FIGS. 4 and 5 are graphs each showing an example of first frequency characteristics that have been Fourier transformed and smoothed in St13. Here, the horizontal axis indicates a frequency and the vertical axis indicates intensity. As can be seen from the example shown in FIG. 4, the intensity of the output signal increases in a frequency bandwidth of from approximately 8 to 13 Hz. The frequency bandwidth corresponds to a frequency bandwidth of an alpha wave specific to the brain wave.

On the other hand, in the example shown in FIG. 5, the increase or the like of the intensity in the specific frequency bandwidth is not seen and the graph falls to the right. In the case where the shape of the graph that is specific to the brain wave is not observed in the first frequency characteristics in this manner, there is a possibility that the output signal has noise because the measurement site and the electrode are electrically disconnected to each other.

In order to determine the electrical connection state between the electrode and the measurement site, the determination unit 15 determines whether or not the first frequency characteristics are similar to the frequency characteristics of pink noise (second frequency characteristics) (St14). The pink noise is noise that generates when the electrical connection between the electrode and the measurement site breaks during a brain wave measurement.

Here, the pink noise is noise having frequency characteristics that the intensity is inversely proportional to the frequency and generally known also as “1/f fluctuation.” Further, the second frequency characteristics are generally expressed by the following expression.

S(f)∝1/f^{α} (S denotes intensity and f denotes frequency (0<α<2))
In this embodiment, assumed is an example of α=½, that is,

S(f)∝1/√f (1).

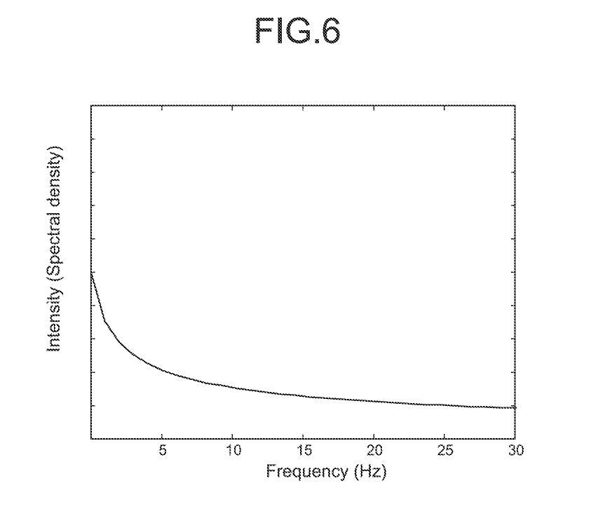

FIG. 6 is a graph showing the second frequency characteristics expressed by Expression (1). Here, the horizontal axis indicates a frequency and the vertical axis indicates intensity. The shape of the graph of the second frequency characteristics shown in FIG. 6 is different from the shape of the graph of the first frequency characteristics shown in FIG. 4. However, the shape of the graph of the second frequency characteristics shown in FIG. 6 is similar to the shape of the graph of the first frequency characteristics shown in FIG. 5. From this, it can be considered that the electrode and the measurement site of the head surface of the user are electrically connected to each other (first state) in the example shown in FIG. 4, while the electrode and the measurement site of the head surface of the user are electrically disconnected to each other (second state) in the example shown in FIG. 5.

A more specific determination is made by calculating similarity between the first frequency characteristics and the second frequency characteristics using a well-known technique.

When the calculated similarity is smaller than a predetermined threshold, that is, when the first frequency characteristics are different from the second frequency characteristics, it is determined that the electrode is in the first state (St14: No). In this case, the measurement unit 16 is caused to continue monitoring while considering the output signal as an appropriate biosignal (brain wave) (St15).

On the other hand, when the calculated similarity is equal to or larger than the predetermined threshold, that is, when the first frequency characteristics are similar to the second frequency characteristics, it is determined that the electrode is in the second state (St14: Yes). In this embodiment, in this case, the storage unit 17 is caused to store the determination result that the electrode is in the second state (St16).

The data stored on the storage unit 17 can be outputted from the output unit 18 to the external apparatus, for example. With this, it is possible to display, in a monitoring result displayed on a screen or the like of the external apparatus, a point of time when it is determined that the electrode is in the second state. With this, it is possible to clearly show the point of time when the electrical connection state at the measurement site is released, and hence to easily check reliability of the data.

As described above, according to this embodiment, based on only the output signal, it is possible to determine the electrical connection state between the measurement site and the electrode. Accordingly, it is possible to determine the connection state while continuing monitoring the output signal. Further, even in the case where it is difficult to make a determination based on the appearance, it is possible to determine the connection state. In addition, without needing a new electrical circuit and the like for determining the connection state, a simple apparatus configuration is achieved.

### Second Embodiment

A second embodiment of the present disclosure will be described.

It should be noted that, in this embodiment, the same configurations as in the first embodiment will be denoted by the same reference symbols and descriptions thereof will be omitted.

[Functional Configuration of Electroencephalograph]

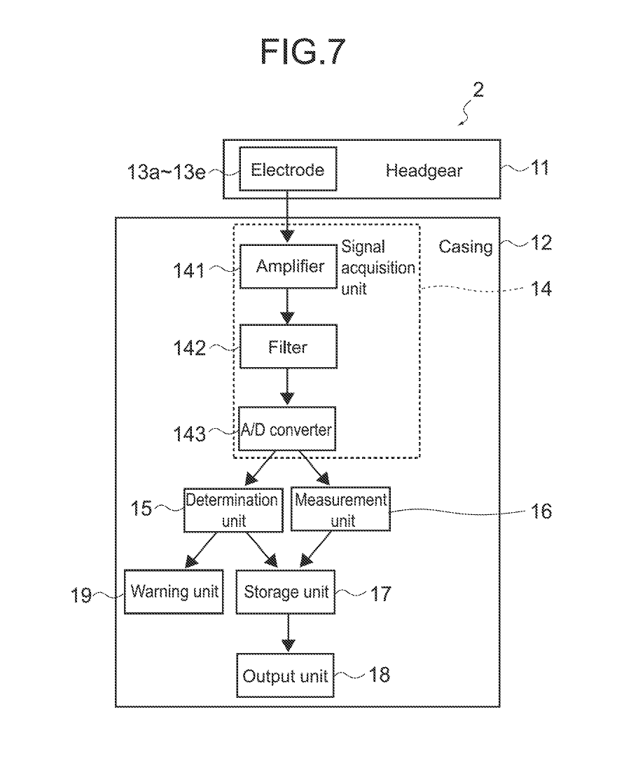

FIG. 7 is a schematic view showing a functional configuration of a biosignal processing apparatus (electroencephalograph) 2 according to this embodiment. As shown in the figure, in addition to a configuration identical to that in the first embodiment, the electroencephalograph 2 includes a warning unit 19. The warning unit 19 is connected to a determination unit 15.

The warning unit 19 may be, for example, an electronic buzzer including a speaker and is provided in the casing 12. When the determination unit 15 determines that the electrode is the second state, the warning unit 19 is activated to warn a measurer or the user of that by, for example, alarming. With this, the measurer or the like can recognize the second state and take an appropriate measure, for example, reconnection.

[Operation of Electroencephalograph]

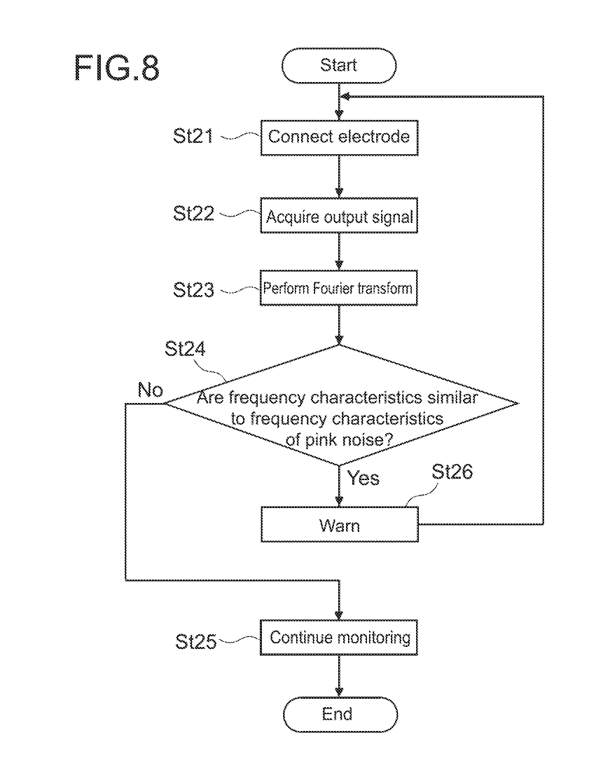

FIG. 8 is a flowchart showing an operation example of the electroencephalograph 2 according to this embodiment.

This embodiment is different from the first embodiment in that, when the first frequency characteristics are similar to the second frequency characteristics and it is determined that at least one of the electrodes 13a to 13e is in the second state (St24: Yes), the warning unit 19 is activated (St26). It should be noted that St21 to St25 shown in the figure correspond to St11 to St15 shown in FIG. 3, respectively, and hence descriptions thereof will be omitted.

When the warning unit 19 is activated, the measurer or the like reconnects the electrodes 13a to 13e to the measurement sites of the head surface of the user, so that the brain wave measurement restarts (St21). If the connection state is not improved, the warning unit 19 is activated again (St26). Therefore, the electrical connection between the measurement site and the electrode can be made secure.

The position of the speaker of the warning unit 19 is not particularly limited and the speaker may be provided to the headgear 11. Alternatively, it is also possible to adopt a configuration of making a notice of which of the electrodes is in the second state by audio or the like. Further, by adopting a configuration in which the warning unit 19 is not directly connected to the determination unit 15 but wiredly or wirelessly connected to the output unit 18, the warning unit 19 itself may be provided to the external apparatus.

### Third Embodiment

A third embodiment of the present disclosure will be described.

It should be noted that, in this embodiment, the same configurations as in the first embodiment will be denoted by the same reference symbols and descriptions thereof will be omitted. [Functional Configuration of Electroencephalograph]

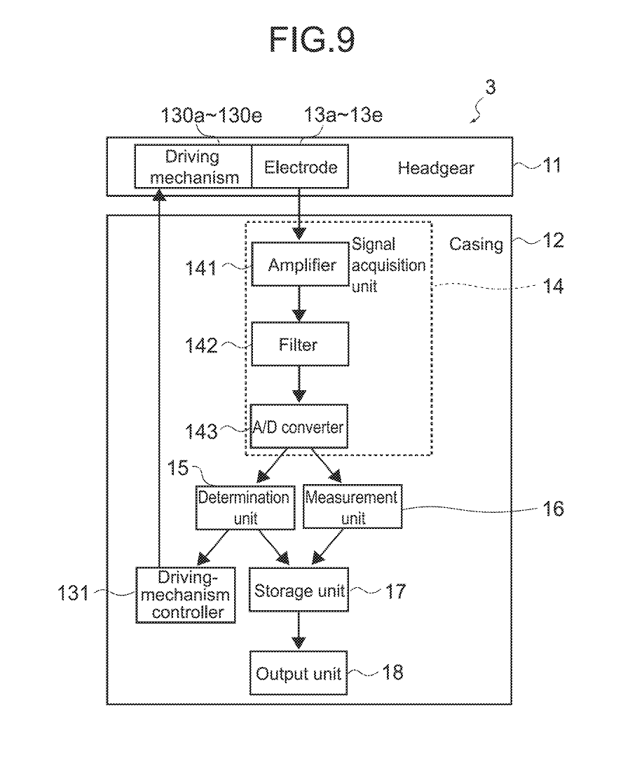

FIG. 9 is a schematic view showing a functional configuration of a biosignal processing apparatus (electroencephalograph) 3 according to this embodiment. As shown in the figure, in addition to a configuration identical to that in the first embodiment, the electroencephalograph 3 includes driving mechanisms 130a to 130e and a driving-mechanism controller 131.

The driving mechanisms 130a to 130e are constituted of motors or the like that are capable of driving each of the electrodes 13a to 13e with respect to a headgear 11. The driving-mechanism controller 131 is connected to a determination unit 15 and the driving mechanisms 130a to 130e and is capable of controlling driving of the driving mechanisms 130a to 130e depending on a determination result of the determination unit 15.

[Operation of Electroencephalograph]

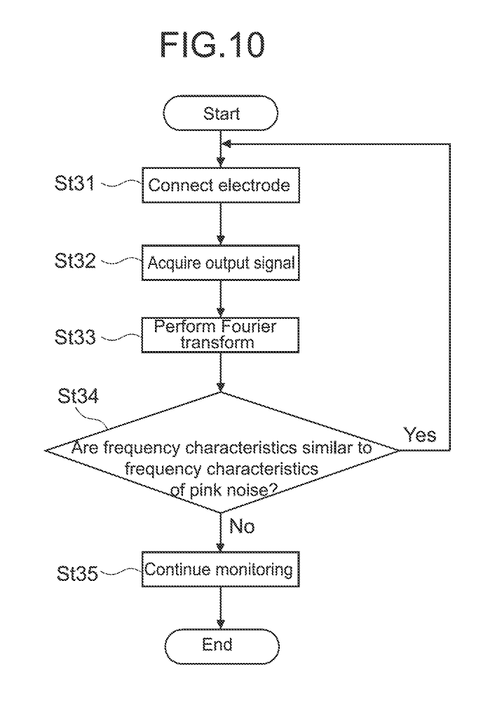

FIG. 10 is a flowchart showing an operation example of the electroencephalograph 3 according to this embodiment.

St31 to St35 shown in the figure correspond to St11 to St15 shown in FIG. 3 and St21 to St25 shown in FIG. 8, respectively. In this embodiment, unlike the second embodiment, when the determination unit 15 determines that any of the electrodes 13a to 13e is in the second state (St34: Yes), the driving-mechanism controller 131 drives the driving mechanism corresponding to that electrode. With this, the electrical connection state is automatically improved. Operations in St31 to St34 when it is determined that the electrode is in the second state are repeated until the determination unit 15 detects the first state (St34: No). It should be noted that, as operations of the electrodes 13a to 13e for improving the connection state, rotation, tilting, or the like with respect to the headgear 11 is adopted.

With the electroencephalograph 3 having the above-mentioned configuration, it is possible to automatically perform processing from determining the electrical connection state between the measurement site and the electrode to establishing the connection state. Accordingly, without needing to monitor the connection state by the measurer or the like, it is possible to correctly measure the brain wave during a long period of time. That is, a very advantageous configuration is achieved even for brain wave measurement during sleep hours or the like.

### Fourth Embodiment

A fourth embodiment of the present disclosure will be described.

It should be noted that, in this embodiment, descriptions of the same configurations as in the first embodiment will be omitted.

A Biosignal Processing Apparatus
(electroencephalograph) according to this embodiment includes a determination unit having a configuration different from that in the first embodiment.

In general, it is known that, when the electrical connection between the measurement site and the electrode breaks, due to the fact that the output signal has the pink noise or the like, the output signal having intensity higher than that of an original brain wave signal generates. For example, comparing FIG. 4 with FIG. 5 described above, it can be seen that the intensity in the second state shown in FIG. 5 is larger than the intensity in the first state shown in FIG. 4 as a whole. Using this, the determination unit according to this embodiment determines whether the electrode is in the first state or in the second state by comparing intensity of the output signal with respect to a specific frequency with a predetermined threshold.

First, as in the first embodiment, the determination unit performs Fourier transform on each of output signals from electrodes to acquire first frequency characteristics. Subsequently, intensity of the first frequency characteristics with respect to the specific frequency is detected and this intensity is compared with the predetermined threshold. Here, when the intensity is smaller than the threshold, it is determined that the electrode is in the first state in which an electrical connection between the measurement site and the electrode is established. Meanwhile, when the intensity is equal to or larger than the threshold, it is determined that the electrode is in the second state in which the electrical connection between the measurement site and the electrode breaks.

As described above, also according to this embodiment, it is possible to easily determine the electrical connection state between the measurement site of the head surface and the electrode. It should be noted that the number of specific frequencies is not limited to one and intensity of the output signal with respect to a plurality of frequencies may be compared with thresholds set corresponding to them, respectively. With this, it is possible to consider a wider frequency bandwidth, and hence to improve reliability of the determination result in the determination unit.

The present disclosure is not limited to the above-mentioned embodiments and modifications can be made without departing from the gist of the present disclosure.

Although, in each of the above-mentioned embodiments, the example in which the biosignal processing apparatus is the electroencephalograph is shown, the present disclosure is not limited thereto. For example, the biosignal processing apparatus does not need to include the headgear and may be used as an electromyography apparatus that measures a myogenic potential as the biosignal. Alternatively, the biosignal processing apparatus may also be used as an electrocardiography apparatus or the like. In these cases, the biosignal processing apparatus may include no electrodes. That is, a configuration in which the biosignal processing apparatus and the electrodes are constituted of separate members and output signals acquired by the electrodes are wirelessly transmitted to the signal acquisition unit may be adopted.

Further, the second frequency characteristics are not limited to the frequency characteristics of the pink noise. Frequency characteristics of noise detected when the electrical connection between the electrode and the measurement site breaks may be used as the second frequency characteristics. Examples of such noise include white noise and colored noise (in addition to pink noise, Brownian noise, etc.). For example, the white noise is noise having the same intensity with respect to all frequencies, and expressed by the following expression:

S_{W}(f)∝1/f⁰ (S_{W} denotes intensity and f denotes frequency). Also in these cases, as in the above-mentioned embodiments, it is possible to determine the electrical connection between the electrode and the measurement site.

Further, although in each of the above-mentioned embodiments, the configuration in which the connection state is determined while the output signal is being monitored is shown, the present disclosure is not limited thereto. A configuration in which the connection state is determined after completion of monitoring may be adopted. In this case, the storage unit stores data on the output signal acquired by the signal acquisition unit and, after the completion of monitoring, the determination unit retrieves the data from the storage unit to determine the connection state. With this configuration, it is possible to analyze the large-volume data after the completion of monitoring.

Although, in the second embodiment, the example in which the warning unit 19 is constituted of the electronic buzzer or the like is shown, the present disclosure is not limited thereto. For example, the warning unit 19 may be constituted of a lighting or blinking circuit using a light-emitting diode (LED) or the like. With this, it is possible to visually warn the measurer or the like of the second state. It should be noted that the position of the LED or the like is not particularly limited and may be provided to the casing 12, the headgear 11, or the external apparatus.

Further, the warning unit 19 may include a vibration motor or the like in order to warn the user of the second state by vibration. Also in this case, the position of the warning unit 19 is not particularly limited.

Further, the present disclosure is applicable not only to biosignals of a human body but also to biosignals of an animal. In order to acquire the biosignals from the animal, it is difficult to determine, in particular, the electrical connection state between the electrode and the measurement site because an individual to measure may be smaller than the human body. With the biosignal processing apparatus according to the embodiments of the present disclosure, it is possible to easily determine the connection state, and hence to acquire more reliable data.

It should be noted that the present disclosure may also take the following configurations.

### (1) A Biosignal Processing Apparatus, Including:

a signal acquisition unit configured to acquire an output signal of an electrode that is placed on a biological surface; and

a determination unit that is connected to the signal acquisition unit to determine, based on first frequency characteristics being frequency characteristics of the output signal, whether the electrode is in a first state in which the electrode is electrically connected to a measurement site of the biological surface or in a second state in which the electrode is electrically disconnected to the measurement site.
(2) The Biosignal Processing Apparatus According to (1), in Which

the determination unit is configured to compare the first frequency characteristics with second frequency characteristics being frequency characteristics of one of colored noise and white noise and to determine, when the first frequency characteristics are different from the second frequency characteristics, that the electrode is in the first state and determine, when the first frequency characteristics are similar to the second frequency characteristics, that the electrode is in the second state.

### (3) The Biosignal Processing Apparatus According to (1), in Which

the determination unit is configured to detect intensity with respect to a specific frequency and to determine, when the intensity is smaller than a predetermined threshold, that the electrode is in the first state and determine, when the intensity is equal to or larger than the threshold, that the electrode is in the second state.

### (4) The Biosignal Processing Apparatus According to Any One of (1) to (3), Further Including

an output unit that is connected to the determination unit to be capable of outputting a determination result of the determination unit.

### (5) The Biosignal Processing Apparatus According to Any One of (1) to (4), Further Including

a warning unit that is connected to the determination unit to be activated when the determination result is that the electrode is in the second state.

### (6) The Biosignal Processing Apparatus According to Any One of (1) to (5), Further Including

a measurement unit configured to monitor, when the determination unit determines that the electrode is in the first state, the output signal as a biosignal over time.

### (7) The Biosignal Processing Apparatus According to Any One of (1) to (6), in Which

the determination unit is configured to perform Fourier transform on the output signal, to thereby acquire the first frequency characteristics.

### (8) A Biosignal Processing Apparatus, Including:

an electrode that is placed on a biological surface;

a signal acquisition unit configured to acquire an output signal of the electrode; and

a determination unit that is connected to the signal acquisition unit to determine, based on first frequency characteristics being frequency characteristics of the output signal, whether the electrode is in a first state in which the electrode is electrically connected to a measurement site of the biological surface or in a second state in which the electrode is electrically disconnected to the measurement site.

### (9) An Electroencephalograph, Including:

a head accessory configured to place an electrode on a head surface of a user;

a signal acquisition unit configured to acquire an output signal of the electrode; and

a determination unit that is connected to the signal acquisition unit to determine, based on first frequency characteristics being frequency characteristics of the output signal, whether the electrode is in a first state in which the electrode is electrically connected to a measurement site of the biological surface or in a second state in which the electrode is electrically disconnected to the measurement site.

### (10) A Biosignal Processing Method, Including:

acquiring an output signal through an electrode for acquiring a biosignal of a biological surface; and

determining, based on first frequency characteristics being frequency characteristics of the output signal, whether the electrode is in a first state in which the electrode is electrically connected to a measurement site of the biological surface or in a second state in which the electrode is electrically disconnected to the measurement site.

### (11) The Biosignal Processing Method According to (10), in Which

the determining includes determining whether the electrode is in the first state or in the second state while monitoring the acquired output signal.

The present disclosure contains subject matter related to that disclosed in Japanese Priority Patent Application JP 2011-224605 filed in the Japan Patent Office on Oct. 12, 2011, the entire content of which is hereby incorporated by reference.

It should be understood by those skilled in the art that various modifications, combinations, sub-combinations and alterations may occur depending on design requirements and other factors insofar as they are within the scope of the appended claims or the equivalents thereof.

What is claimed is:

1. A biosignal processing apparatus, comprising:
a signal acquisition unit configured to acquire an output signal of an electrode that is placed on a biological surface; and
a determination unit that is connected to the signal acquisition unit to determine, based on first frequency characteristics being frequency characteristics of the output signal, whether the electrode is in a first state in which the electrode is electrically connected to a measurement site of the biological surface or in a second state in which the electrode is electrically disconnected to the measurement site.

2. The biosignal processing apparatus according to claim 1, wherein
the determination unit is configured to compare the first frequency characteristics with second frequency characteristics being frequency characteristics of one of colored noise and white noise and to determine, when the first frequency characteristics are different from the second frequency characteristics, that the electrode is in the first state and determine, when the first frequency characteristics are similar to the second frequency characteristics, that the electrode is in the second state.

3. The biosignal processing apparatus according to claim 1, wherein
the determination unit is configured to detect intensity with respect to a specific frequency and to determine, when the intensity is smaller than a predetermined threshold, that the electrode is in the first state and determine, when the intensity is equal to or larger than the threshold, that the electrode is in the second state.

4. The biosignal processing apparatus according to claim 1, further comprising
an output unit that is connected to the determination unit to be capable of outputting a determination result of the determination unit.

5. The biosignal processing apparatus according to claim 1, further comprising
a warning unit that is connected to the determination unit to be activated when the determination result is that the electrode is in the second state.

6. The biosignal processing apparatus according to claim 1, further comprising
a measurement unit configured to monitor, when the determination unit determines that the electrode is in the first state, the output signal as a biosignal over time.

7. The biosignal processing apparatus according to claim 1, wherein
the determination unit is configured to perform Fourier transform on the output signal, to thereby acquire the first frequency characteristics.

8. A biosignal processing apparatus, comprising:
an electrode that is placed on a biological surface;
a signal acquisition unit configured to acquire an output signal of the electrode; and
a determination unit that is connected to the signal acquisition unit to determine, based on first frequency characteristics being frequency characteristics of the output signal, whether the electrode is in a first state in which the electrode is electrically connected to a measurement site of the biological surface or in a second state in which the electrode is electrically disconnected to the measurement site.

9. An electroencephalograph, comprising:
a head accessory configured to place an electrode on a head surface of a user;
a signal acquisition unit configured to acquire an output signal of the electrode; and
a determination unit that is connected to the signal acquisition unit to determine, based on first frequency characteristics being frequency characteristics of the output signal, whether the electrode is in a first state in which the electrode is electrically connected to a measurement site of the biological surface or in a second state in which the electrode is electrically disconnected to the measurement site.

10. A biosignal processing method, comprising:
acquiring an output signal through an electrode for acquiring a biosignal of a biological surface; and
determining, based on first frequency characteristics being frequency characteristics of the output signal, whether the electrode is in a first state in which the electrode is electrically connected to a measurement site of the biological surface or in a second state in which the electrode is electrically disconnected to the measurement site.

11. The biosignal processing method according to claim 10, wherein
the determining includes determining whether the electrode is in the first state or in the second state while monitoring the acquired output signal.
